(19) United States
(12) Patent Application Publication
(10) Pub. No.: US 2003/0162209 A1
(43) Pub. Date: Aug. 28, 2003

(54) PCR based high throughput polypeptide screening

(76) Inventor: George Martin, Berkeley, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/327,292

(22) Filed: Dec. 19, 2002

Related U.S. Application Data
(60) Provisional application No. 60/341,978, filed on Dec. 19, 2001.

(51) Int. Cl. C12Q 1/68; G01N 33/53; G01N 33/567; C12P 21/02; C12N 5/06; C12N 15/85

(52) U.S. Cl. 435/6; 435/7.2; 435/455; 435/325; 435/320.1

(57) ABSTRACT

High throughput screening of polypeptides and the replication of the corresponding genetic coding sequences is accomplished by amplification of an initial polynucleotide template or library of templates. The amplification product is used as a template for coupled in vitro transcription and translation. The translation product is then screened for a property of interest, e.g. binding specificity, enzymatic activity, substrate specificity, and the like. Polynucleotide sequences encoding a desired polypeptide are directly transformed into a host cell for further screening, replication, rounds of selection, and the like. The initial template, or library of templates may be mutagenized to generate a plurality of sequence variants for screening.

### BACKGROUND OF THE INVENTION

The generation of large libraries for in vitro protein testing presents the challenge of effectively screening libraries containing large numbers of sequence variants on the basis of their biological properties, such as binding, catalytic activity, specificity, and the like. The explosion in numbers of potential new targets resulting from genomics and combinatorial chemistry approaches over the past few years has placed enormous pressure on screening programs. While the rewards for identification of a useful change can be great, the percentage of hits is typically low. To address this problem, screening methods that can provide for a high throughput method are preferable, so that many individual polypeptides can be tested.

In vitro protein synthesis is as an effective tool for lab-scale expression of cloned or synthesized genetic materials. In recent years, in vitro protein synthesis has greatly augmented conventional recombinant DNA technology, because of disadvantages associated with cellular expression. In vivo, proteins can be degraded or modified by several enzymes synthesized with the growth of the cell, and after synthesis may be modified by post-translational processing, such as glycosylation, deamination or oxidation. In addition, many products inhibit metabolic processes and their synthesis must compete with other cellular process required to reproduce the cell and to protect its genetic information. Further, in vitro protein synthesis systems have added flexibility compared to in vivo systems. For example, additives known to enhance protein solubility and activity e.g, chaperones, detergents and cofactors and the like are easily included during the synthesis of the target polypeptide. The simultaneous expression of multiple proteins is also much more easily accomplished in cell-free systems. Methods of in vitro transcription and translation are described, for example, in U.S. Pat. No. 6,168,931; U.S. Pat. No. 6399323; Kim and Swartz (2000) Biotechnol Prog. 16:385-390; Kim and Swartz (2000) Biotechnol Lett. 22:1537-1542; Kim and Choi (2000) J Biotechnol. 84:27-32; Kim et al. (1996) Eur J Biochem. 239: 881-886; Kim and Swartz (2001) Biotechnol Bioeng. 74:309-316; and Kim and Swartz (1999) Biotechnol Bioeng. 66:180-188, herein incorporated by reference.

While in vitro protein synthesis provides a convenient format for screening, current methods for altering gene sequences usually employ a step whereby plasmids are propagated in bacteria. Even methods that utilize PCR to generate DNA fragments to direct the production of mutated proteins rely on a process known as overlap extension PCR. Overlap extension PCR has the disadvantage that the PCR product must be cloned after it has been discovered to encode a protein with the desired characteristics.

Methods that streamline the high throughput screening of polypeptides encoded by amplification products are of great interest for the development of novel polypeptide agents. This issue is addressed by the present invention.

References of interest include U.S. Pat. Nos. 5,545,552; 5,789,166; 5,866,395; 5,923,419; 5,948,663; and 6,183,997. Other publications of interest include Ohuchi et al. (1998) N.A.R. 26:4339-4346; Garvin et al. (2000) Nat. Biotech. 18:95-97; and Lee and Cohen (2001) J. Biol. Chem. 276:23268-23274. U.S. Pat. No. 6,280,977 describes a method of overlap extension PCR.

### SUMMARY OF THE INVENTION

Methods are provided for high throughput screening of sequences comprising an expressible open reading frame. An expressible portion of an initial replicatible template is amplified, e.g. by PCR amplification. Such an expressible portion comprises an open reading frame operably linked to regulatory elements for transcription and translation. The resulting amplification product is then used as a template for expression by coupled in vitro transcription and translation. The resulting polypeptide is screened for a property of interest, e.g. binding specificity, enzymatic activity, substrate specificity, and the like. Initial templates encoding a translation product of interest are used directly to transform a host cell, without an intervening cloning step. In this way, the process of obtaining and replicating sequences encoding a polypeptide of interest is streamlined.

In one embodiment of the invention, the initial template is a product of a ligation or recombination reaction, where the reactants are linear molecules and the product is a circular molecule. Primers may be selected that only provide for exponential amplification of the circular molecule.

In one embodiment of the invention, the amplification primers will not hybridize to a complete promoter sequence. In another embodiment of the invention, the amplification primer comprises a terminal GC clamp region.

In one embodiment of the invention the initial template comprises methylated nucleotides, and prior to expression, the product of the amplification reaction is digested with a restriction enzyme specific for methylated nucleotides.

### BRIEF DESCRIPTION OF THE DRAWINGS

FIG. 1 illustrates the generation of expressible PCR products from circular molecules.

FIG. 2 depicts amplification of linear and circular molecules, where the primers only provide for exponential amplification of the circular molecule.

FIGS. 3A and 3B depict the results of site directed mutagenesis.

FIG. 4 depicts the PCR products, and translation products, after amplification of ligation reactions.

FIG. 5 is a graph depicting the result of expression of amplification products from a recombinational cloning reaction.

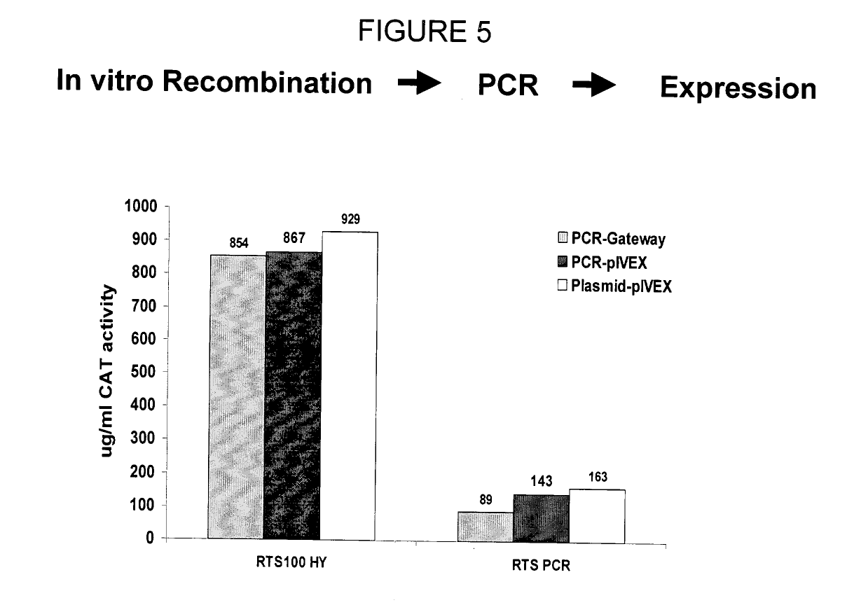

### DETAILED DESCRIPTION OF THE EMBODIMENTS

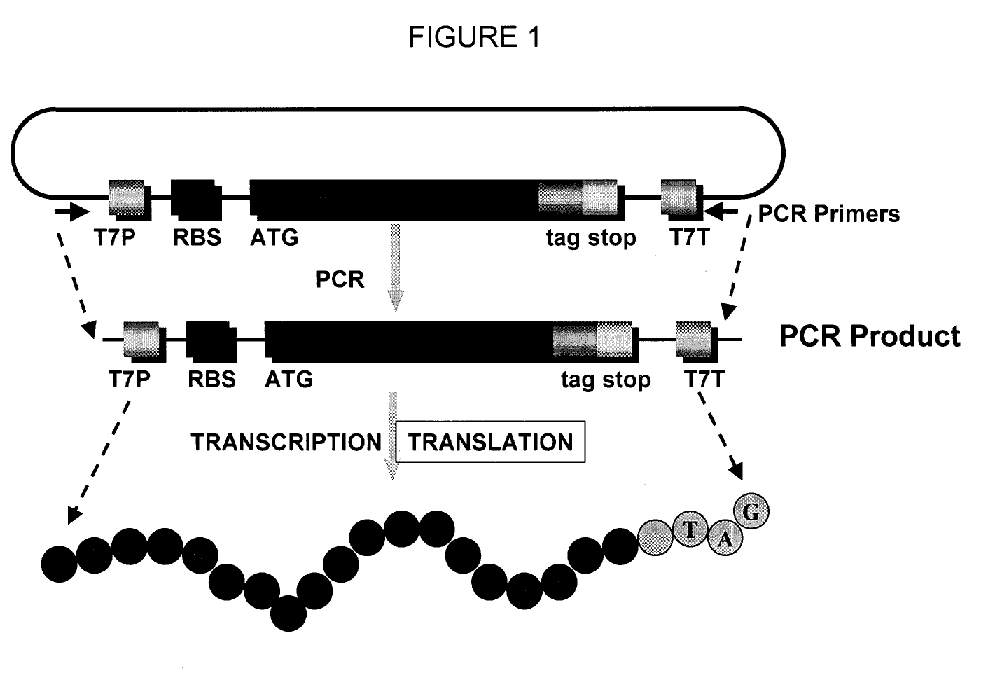

Methods for the streamlined high throughput screening of polypeptides and the replication of the corresponding genetic coding sequences are provided. In the methods of the invention, an expressible portion of an initial replicatible polynucleotide template or library of templates is amplified, e.g. by PCR, as shown in FIG. 1. The initial template, or library of templates may be mutagenized to generate a plurality of sequence variants for screening. The expressible portion of the template comprises an open reading frame operably joined to regulatory sequences for transcription and translation. The expressible portion may comprise a fragment of the template, up to and including the complete replicatible molecule.

The amplification product is used as a template for coupled in vitro transcription and translation to express the product of the open reading frame. The amplification reaction may be used directly for expression in the absence of additional purification steps to isolate the amplification product. The polypeptide translation product is then screened for a property of interest, e.g. binding specificity, enzymatic activity, substrate specificity, and the like.

Initial templates comprising an open reading frame encoding a product having a property of interest are then directly transformed into a host cell, without an intervening cloning step. The term “intervening cloning step” is intended to refer to the ligation or recombination of a sequence of interest with a second polynucleotide, e.g. ligation of a polynucleotide into a vector, etc. In this way, the process of obtaining and replicating sequences encoding a polypeptide of interest is streamlined.

An additional amplification step is optionally performed, for example when the initial template is present in very small quantities, where the complete replicatible molecule is amplified prior to transformation.

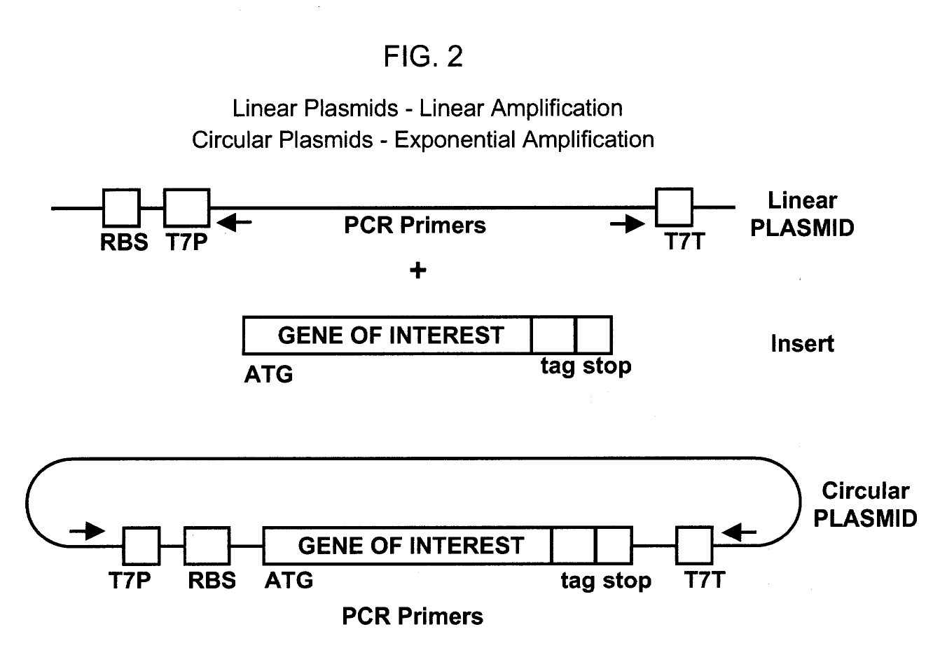

The initial template may be a product of a ligation or recombination reaction, where the reactants are linear molecules and the product is a circular molecule. Primers may be selected that only provide for exponential amplification of an expressible portion of the circular molecule, as shown in FIG. 2. A first and a second primer, P1 and P2, are selected to hybridize to the linear vector, but prime away from each other on the linear molecule. The linear molecule is therefore unable to generate exponential increases in the replication product during rounds of amplification. It is only when the complete circular molecule is formed that a “bridge” is created between the two primers, such that they prime towards each other.

Usually one of the amplification primers will hybridize to a region of the initial template at, or upstream, of a promoter for the expressible portion, (herein designated P1 for convenience). Preferably the primer will not hybridize to a complete promoter sequence, e.g. hybridizing upstream of the promoter, or comprising a partial promoter sequence. Such primers find particular use when it is desirable to express the amplification product directly from the amplification reaction without intervening purification steps. The P1 primer optionally comprises a GC clamp region at the 5′ terminus, which stabilizes the DNA template. The ends of PCR fragments are prone to digestion by exonucleases, e.g. during the transcription reaction. The GC clamp region does not hybridize to a target sequence, but protects the 5′-ends from exonuclease digestion.

In one embodiment of the invention, PCR based mutagenesis is performed on a template comprising methylated nucleotides. Following the PCR based mutagenasis, the product of the mutagenesis reaction is digested with a restriction enzyme specific for methylated nucleotides, which cleaves the methylated parent DNA, but does not cleave the mutagenized product. The mutagenized product is then amplified and expressed in accordance with the methods of the invention.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention, which will be limited only by the appended claims.

As used herein the singular forms “a”, “and”, and “the” include plural referents unless the context clearly dictates otherwise. Thus, for example, reference to “a cell” includes a plurality of such cells and reference to “the protein” includes reference to one or more proteins and equivalents thereof known to those skilled in the art, and so forth. All technical and scientific terms used herein have the same meaning as commonly understood to one of ordinary skill in the art to which this invention belongs unless clearly indicated otherwise.

The initial polynucleotide template is a replicatible molecule. As used herein, the term refers to polynucleotide molecules, usually double stranded DNA and frequently circular, that are capable of replicating when transformed into a host cell. Minimally such polynucleotides will comprise an origin of replication active on the desired host cell, e.g. an origin of replication active in a bacterial cell, and origin of replication active in an animal cell, an origin of replication active in a fungal cell, including yeast cells, an origin of replication active in a plant cell, and the like. Often such polynucleotides will further comprise one or more selectable markers, e.g. drug resistance, expression of a recombinase gene, expression of a fluorescent or otherwise detectable gene product, and the like. Such sequences are well known in the art.

The initial polynucleotide template further comprises an expressible portion comprising sequences that are expressed with in vitro transcription and translation systems. Elements of the expressible portion include a promoter element, e.g. T7 promoter; T3 promoter; SP6 promoter; etc. Mammalian promoters, e.g. CMV promoter, may also find use. Also included is a ribosome binding site; an initiation codon; and a coding sequence of interest, i.e. an open reading frame. Optionally included elements are a stop codon; and transcription termination sequence.

The coding sequence of interest can be obtained from any of a variety of sources or methods well known in the art, e.g. isolated from suitable cells, produced using synthetic techniques, etc., and the constructs prepared using recombinant techniques well known in the art. Sequences of many gene products desirable for analysis according to the method of the invention are known. Such sequences have been described in the literature, are available in public sequence databases such as GenBank, or are otherwise publicly available. With the availability of automated nucleic acid synthesis equipment, both DNA and RNA can be synthesized directly when the nucleotide sequence is known, or synthesized by PCR cloning followed by growth in a suitable microbial host. Moreover, when the amino acid sequence of a desired polypeptide is known, a suitable coding sequence for the nucleic acid can be inferred. Where the DNA encoding a gene product of interest has not been isolated, this can be accomplished by various, standard protocols well known to those of skill in the art (see, for example, Sambrook et al., ibid; Suggs et al. 1981 Proc. Natl. Acad. Sci. USA 78:6613-6617; U.S. Pat. No. 4,394,443; each of which are incorporated herein by reference with respect to identification and isolation of DNA encoding a gene product of interest).

Sequences of interest include, for example, genetic sequences of pathogens; genes encoding enzymes, e.g. proteases, kinases, polymerases, etc.; genes encoding antigens; genes involved in drug resistance; and the like; for example coding regions of viral, bacterial protozoan, plant and animal genes, coding sequences for antibodies or single chain antibodies, and the like. Sequences from two or more sequences may recombined or shuffled to provide hybrid sequences. A large number of public resources are available as a source of genetic sequences, e.g. for human, other mammalian, and human pathogen sequences. A substantial portion of the human genome is sequenced, and can be accessed through public databases such as Genbank. Resources include the uni-gene set, as well as genomic sequences. For example, see Dunham et al. (1999) Nature 402, 489-495; or Deloukas et al. (1998) Science 282, 744-746. For example, cDNA clones corresponding to many human gene sequences are available from the IMAGE consortium. The international IMAGE Consortium laboratories develop and array cDNA clones for worldwide use. The clones are commercially available, for example from Genome Systems, Inc., St. Louis, Mo. Methods for cloning sequences by PCR based on DNA sequence information are also known in the art.

Likewise, techniques for inserting regulatory sequences required for expression are known in the art (see, for example, Kormal et al., Proc. Natl. Acad. Sci. USA, 84:2150-2154, 1987; Sambrook et al. Molecular Cloning: A Laboratory Manual, 2nd Ed., 1989, Cold Spring Harbor Laboratory Press, Cold Spring Harbor, N.Y.; each of which are hereby incorporated by reference with respect to methods and compositions for expression of a sequence of interest).

A library of initial templates may be utilized for amplification. Such a library may be obtained by in vitro mutagenesis of a sequence of interest, by in vivo mutagenesis, e.g. followed by selection for a trait of interest, and the like, as known in the art. Shuffling of sequences may also be used to generate mutations. For example, see U.S. Pat. No. 6,479,652, “Oligonucleotide mediated nucleic acid recombination”; U.S. Pat. No. 6,455,253, “Methods and compositions for polypeptide engineering”; U.S. Pat. No. 6 6,413,745, “Recombination of insertion modified nucleic acids”; and U.S. Pat. No. 6,352,859, “Evolution of whole cells and organisms by recursive sequence recombination”, herein incorporated by reference.

A “library” refers to a collection, or plurality, of polynucleotides. A particular library might include, for example, templates comprising different site specific mutations, a collection of random mutations in a coding sequence of interest, shuffled sequences, etc. In the methods of the present invention, polynucleotides in the library are typically spatially separated, for example one clone per well of a microtiter plate. When a reaction, e.g. an amplification reaction, a transcription and translation reaction, etc. is performed on a spatially separated library, the same reaction is usually performed separately on every member of the library.

In one embodiment of the invention, amplification is used to mutagenize sequences to generate a library of sequence variants, which variant sequences are screened for characteristics of interest, e.g. enhanced binding, thermal or pH stability, emission of specific light spectra, enzymatic activity and specificity, and the like. Such a mutagenesis may be site directed, or random, or may combine elements of both, i.e. a random introduction of nucleotides at a specific site, and the like.

Reactions for in vitro mutagenesis typically are based on a nucleic acid template that comprises a sequence of interest. The template is used to generate altered copies, where the alteration may be site-specific, randomly located, or a combination thereof. Templates may be double stranded or single stranded, linear or circular, and may be DNA, RNA, or a synthetic analog thereof. In one embodiment of the invention, a methylated template is used, which can be cleaved after the mutagenesis reaction with an enzyme specific for methylated residues, e.g. DPNI, which selectively cleaves only methylated DNAs. For a review of mutagenesis methods, see Ling and Robinson (1997) Anal. Biochem. 254:157-178, herein incorporated by reference.

Strategies include site directed mutagenesis, where a specific mutagenic primer is used, resulting in a specific mutant with a predetermined site and type of mutation. For examples, “scanning” mutations are used to introduce a single codon change, e.g. an alanine substitution, along the length of a protein. To rapidly generate multiple changes at a targeted site, degenerate primers may be used, in order to increase the number of possible mutations from a single reaction. Alternatively, a set of random mutations over a region or an entire gene is desired, random and extensive mutagenesis may be used.

Mutagenesis may include the introduction of specific mutations or combinations of mutations into a primer, where the primer contains sufficient homology to anneal to a site on the nucleic acid template, but where there is not a perfect match between the primer and the template, i.e. the primer contains one, two, three or more mutagenized positions. The introduced mutations may be pre-determined, where specific residues are introduced into the sequence, or may comprise a random mixture, e.g. where one, two, three or more positions in the primer are synthesized with a random mixture of nucleotides. For example, the three nucleotides corresponding to a specific codon may be randomly mutagenized. Other mutagenesis methods of interest include insertions or deletions at any location within the coding sequence.

Typically the primer will be free of strong secondary structure, such as hairpins, loops or direct repeats. The mismatched, or mutagenized residues, are often located towards the middle of the primer, rather than at the termini, although inverse PCR and ligation PCR preferably place the mutation at the 5′ terminus.

Conveniently, PCR is used to generate the mutagenized nucleic acid. For example, a primer containing mutagenized residues may be used as an amplification primer in a PCR reaction. Where the primer contains multiple mutations, the mutagenesis reaction may be a single, or small number of cycles of amplification, where the mutagenized product is then used as a template for further amplification with non-mutagenized primers. The selection of enzyme for the amplification reaction will be determined by the requirement for fidelity, where enzymes such a Taq polymerase typically introduce a higher number of random mutations, and enzymes such as Pfu, or Tgo or blended combinations of polymerases increase the fidelity of the reaction. Error-prone PCR uses low-fidelity polymerization conditions to introduce a low level of point mutations randomly over a long sequence.

The polynucleotide sequence can also be altered by chemical mutagenesis. Chemical mutagens include, for example, sodium bisulfite, nitrous acid, hydroxylamine, hydrazine or formic acid. Other agents that are analogues of nucleotide precursors include nitrosoguanidine, 5-bromouracil, 2-aminopurine, or acridine. Generally, these agents are added to the PCR reaction in place of the nucleotide precursor thereby mutating the sequence. Intercalating agents such as proflavine, acriflavine, quinacrine and the like can also be used. Random mutagenesis of the polynucleotide sequence can also be achieved by irradiation with X-rays or ultraviolet light.

Non-PCR reactions may also be used to for mutagenesis, where similar selection of template, primers and nucleotides are used, but in a conventional synthesis reaction. Enzymes may be thermolabile for non-PCR mutagenesis, e.g. Klenow, T7 DNA polymerase, T4 DNA polymerase, and the like. Alternatively, in vivo methods of mutagenesis may be used, for example in combination with an initial selection for a trait of interest.

An expressible portion of the initial template or library of templates is amplified to produce sufficient polynucleotides for transcription and translation analysis. As described above, the amplification primers may be selected to differentiate between linear and circular reactants of a ligation or recombination reaction, as shown in FIG. 2. The primers are selected to hybridize to the linear polynucleotide, but to prime away from each other. The linear molecule is therefore unable to generate exponential increases in the replication product during rounds of amplification. When an open reading frame of interest is recombinaed or ligated into the vector backbone a “bridge” is created between the two primers, such that they prime towards each other.

Ampllification primers may also be selected to comprise a terminal GC clamp. A GC clamp comprises at least about 5 and not more than about 10 GC residues, e.g. alternating GC residues or a homopolymer of either G or C. The GC clamp region usually does not hybridize to a sequence present on the template.

When oriented with respect to the coding sequence, the upstream, or 5′ amplification primer will hybridize to a region of the initial template at, or upstream, of a promoter for the expressible portion. To avoid, for example, competition for RNA polymerase during a subsequent coupled transcription/translation reaction, it is preferable that the primer will not comprise to a complete promoter sequence, e.g. it will hybridize upstream of the promoter, or will comprise only a partial promoter sequence.

The term “amplify” in reference to a polynucleotide means to use any method to produce multiple copies of a polynucleotide segment, called the “amplicon” or “amplification product”, by replicating a sequence element from the polynucleotide or by deriving a second polynucleotide from the first polynucleotide and replicating a sequence element from the second polynucleotide. The copies of the amplicon may exist as separate polynucleotides or one polynucleotide may comprise several copies of the amplicon. The precise usage of amplify is clear from the context to one skilled in the art.

A preferred amplification method utilizes PCR (see Saiki et al. (1988) Science 239:487-4391). Briefly, the method as now commonly practiced utilizes a pair of primers that have nucleotide sequences complementary to the DNA which flanks the target sequence. The primers are mixed with a solution containing the target DNA (the template), a thermostable DNA polymerase and deoxynucleoside triphosphates (dNTPS) for all four deoxynucleotides. The mix is then heated to a temperature sufficient to separate the two complementary strands of DNA. The mix is next cooled to a temperature sufficient to allow the primers to specifically anneal to sequences flanking the gene or sequence of interest. The temperature of the reaction mixture is then optionally reset to the optimum for the thermostable DNA polymerase to allow DNA synthesis (extension) to proceed. The temperature regimen is then repeated to constitute each amplification cycle. Thus, PCR consists of multiple cycles of DNA melting, annealing and extension. Twenty replication cycles can yield up to a million-fold amplification of the target DNA sequence. In some applications a single primer sequence functions to prime at both ends of the target, but this only works efficiently if the primer is not too long in length. In some applications several pairs of primers are employed in a process commonly known as multiplex PCR.

The PCR methods used in the methods of the present invention are carried out using standard methods (see, e.g., McPherson et al., PCR (Basics: From Background to Bench) (2000) Springer Verlag; Dieffenbach and Dveksler (eds) PCR Primer: A Laboratory Manual (1995) Cold Spring Harbor Laboratory Press; Erlich, PCR Technology, Stockton Press, New York, 1989; Innis et al., PCR Protocols: A Guide to Methods and Applications, Academic Press, Harcourt Brace Javanovich, New York, 1990; Barnes, W. M. (1994) Proc Natl Acad Sci USA, 91, 2216-2220). The primers and oligonucleotides used in the methods of the present invention are preferably DNA and analogs thereof, e.g. phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH₂-5′-O-phosphonate and 3′-NH-5′-O-phosphoroamidate. Such nucleic acids can be synthesized using standard techniques

The number of cycles of amplification will generate sufficient polynucleotide product to analyze an aliquot in an in vitro transcription and translation reaction, and to provide sufficient polynucleotide for transformation, if desired. Typically at least about 10 cycles, at least about 15 cycles, at least about 20 cycles, at least about 30 cycles or more will be utilized. The number of cycles for a particular application will be determined by the amount of initial template present, the requirements for transformation into a host, the protein screening and transcription/translation efficiency, and the like.

An aliquot of the amplification product is used as a template for in vitro transcription and translation, preferably in a high throughput format, e.g. an array of microtiter wells, or the like. In vitro synthesis as used herein refers to the cell-free synthesis of polypeptides in a reaction mix comprising biological extracts and/or defined reagents. The reaction mix will comprise at least ATP, an energy source; a template for production of the macromolecule, e.g. DNA, mRNA, etc.; amino acids, and such co-factors, enzymes and other reagents that are necessary for the synthesis, e.g. ribosomes, tRNA, polymerases, transcriptional factors, etc. Such synthetic reaction systems are well-known in the art, and have been described in the literature. The cell free synthesis reaction may be performed as batch, continuous flow, or semi-continuous flow, as known in the art, preferably in a high throughput batch format.

For the purposes of this invention, biological extracts are any preparation comprising the components of protein synthesis machinery, usually a cell extract, wherein such components are capable of expressing a nucleic acid encoding a desired protein. Thus, a cell extract comprises components that are capable of translating messenger-ribonucleic acid (mRNA) encoding a desired protein, and optionally comprises components that are capable of transcribing DNA encoding a desired protein. Such components include, for example, DNA-directed RNA polymerase (RNA polymerase), any transcription activators that are required for initiation of transcription of DNA encoding the desired protein, transfer ribonucleic acids (tRNAs), aminoacyl-tRNA synthetases, 70S ribosomes, N¹⁰-formyltetrahydrofolate, formylmethionine-tRNAf^{Met} synthetase, peptidyl transferase, initiation factors such as IF-1, IF-2 and IF-3, elongation factors such as EF-Tu, EF-Ts, and EF-G, release factors such as RF-1, RF-2, and RF-3, and the like.

In a preferred embodiment of the invention, the reaction mixture comprises extracts from biological sources, e.g. E. coli S30 extracts, wheat germ extracts, reticulocyte extracts, etc., as is known in the art. For convenience, the organism used as a source of extracts may be referred to as the source organism. Methods for producing active extracts are known in the art, for example they may be found in Pratt (1984), Coupled transcription-translation in prokaryotic cell-free systems, p. 179-209, in Hames, B. D. and Higgins, S. J. (ed.), Transcription and Translation: A Practical Approach, IRL Press, New York. Kudlicki et al. (1992) Anal Biochem 206(2):389-93 modify the S30 E. coli cell-free extract by collecting the ribosome fraction from the S30 by ultracentrifugation.

The reactions are preferably small scale, and may be multiplexed to perform a plurality of simultaneous syntheses. Continuous reactions will use a feed mechanism to introduce a flow of reagents, and may isolate the end-product as part of the process. Batch systems are also of interest, where additional reagents may be introduced to prolong the period of time for active synthesis.

In addition to the above components such as cell-free extract, genetic template, amino acids and energy sources, materials specifically required for protein synthesis may be added to the reaction. These materials include salt, polymeric compounds, cyclic AMP, inhibitors for protein or nucleic acid degrading enzymes, inhibitor or regulator of protein synthesis, oxidation/reduction adjuster, non-denaturing surfactant, buffer component, spermine, spermidine, etc.

The salts preferably include potassium, magnesium, ammonium and manganese salt of acetic acid or sulfuric acid, and some of these may have amino acids as a counter anion. The polymeric compounds may be polyethylene glycol, dextran, diethyl aminoethyl, quaternary aminoethyl and aminoethyl. The oxidation/reduction adjuster may be dithiothreitol, ascorbic acid, glutathione and/or their oxides. Also, a non-denaturing surfactant such as Triton X-100 may be used at a concentration of 0-0.5 M. Spermine and spermidine may be used for improving protein synthetic ability, and cAMP may be used as a gene expression regulator. Preferably, the reaction is maintained in the range of pH 5-10 and a temperature of 20°-50° C., and more preferably, in the range of pH 6-9 and a temperature of 25°-40° C.

The amount of protein produced in a translation reaction can be measured in various fashions. One method relies on the availability of an assay, which measures the activity of the particular protein being translated. Another method of measuring the amount of protein produced in coupled in vitro transcription and translation reactions is to perform the reactions using a known quantity of radiolabeled amino acid such as ³⁵S-methionine or ³H-leucine and subsequently measuring the amount of radiolabeled amino acid incorporated into the newly translated protein. Incorporation assays will measure the amount of radiolabeled amino acids in all proteins produced in an in vitro translation reaction including truncated protein products. The radiolabeled protein may be further separated on a protein gel, and by autoradiography confirmed that the product is the proper size and that secondary protein products have not been produced.

The polypeptide produced in a translation reaction is screened for a property of interest, including stability, e.g. to pH, ionicity, temperature, radiation, and the like; specificity, e.g. substrate specificity, receptor binding specificity, ligand specificity, and the like; enzymatic activity, e.g. rate of catalysis, product, and the like; etc. The specific screening format will be designed based on the polypeptide and its properties.

A reaction can be conducted in a liquid phase, the reaction products separated from unreacted components, and products detected; e.g. using an immobilized antibody specific for the gene product or the test compound to anchor any complexes formed in solution, and a labeled antibody specific for the other component of the possible complex to detect anchored complexes.

Alternatively, the polypeptide may be anchored onto a solid surface, and the product of the screening, e.g. binding complex, reaction product, etc. may be detected on the solid phase or the supernatant. In practice, microtiter plates may conveniently be utilized as the solid phase. The anchored component may be immobilized by non-covalent or covalent attachments. Non-covalent attachment may be accomplished by simply coating the solid surface with a solution of the protein and-drying. Alternatively, an immobilized antibody specific for the protein to be immobilized may be used to anchor the protein to the solid surface. The non-immobilized component is added to the coated surface containing the anchored component. After the reaction is complete, unreacted components are removed (e.g., by washing) under conditions such that any products formed remain immobilized on the solid surface; or are detected in the supernatant.

For example, if a polynucleotide that encodes a protein with increased binding efficiency to a ligand is desired, the proteins expressed from each of the expressible portions of the polynucleotides in the population or library may be tested for their ability to bind to the ligand by methods known in the art (i.e. panning, affinity chromatography). If a polynucleotide that encodes for a protein with increased drug resistance is desired, the proteins expressed by each of the polynucleotides in the population or library may be tested for their ability to confer drug resistance, e.g. cleavage of β-lactam, and the like. One skilled in the art, given knowledge of the desired protein, could readily test the population to identify polynucleotides that confer the desired properties onto the protein.

An initial template(s) comprising an expressible portion of interest, i.e. a portion that encodes a polypeptide having a desired property, is directly transformed into a host for further replication, in the absence of an intervening cloning step. Optionally a replicatible portion of the initial template is amplified prior to transformation.

Generally an aliquot of the initial template is used for the polypeptide expression and screening, and the remaining sample maintained for transformation or further manipulation, if desired. Methods of transformation are well known in the art. Preferred host cells include E. coli, B. subtilis, S. cerevisiae, insect cells in combination with baculovirus vectors, or cells of a higher organism such as vertebrates, particularly mammals, e.g. COS 7 cells, or 293 cells.

A number of cycles of mutagenesis, amplification, screening and transformation may be conducted. In this manner, proteins with even higher binding affinities, enzymatic activity, increased solubility, stability etc. may be achieved.

The reagents utilized in the methods of the invention may be provided in a kit, which kit may further include instructions for use. Such a kit may comprise, for example: a vector for use in generating initial templates; reagents for mutagenesis; reagents for amplification; reagents for in vitro transcription and translation; and host cells for transformation. The term reagents may include: buffers; enzymes; monomers, e.g. nucleotide triphosphates, amino acids, and the like; polynucleotide sequences, e.g. polynucleotide primers, control templates, vector sequences, etc. The kit reagents may be provided with container suitable for parallel, high throughput screening, e.g. 96 well plates, and the like.

It is to be understood that this invention is not limited to the particular methodology, protocols, cell lines, animal species or genera, constructs, and reagents described, as such may, of course, vary. It is also to be understood that the terminology used herein is for the purpose of describing particular embodiments only, and is not intended to limit the scope of the present invention, which will be limited only by the appended claims.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing, for example, the cell lines, constructs, and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### EXPERIMENTAL

### Example 1

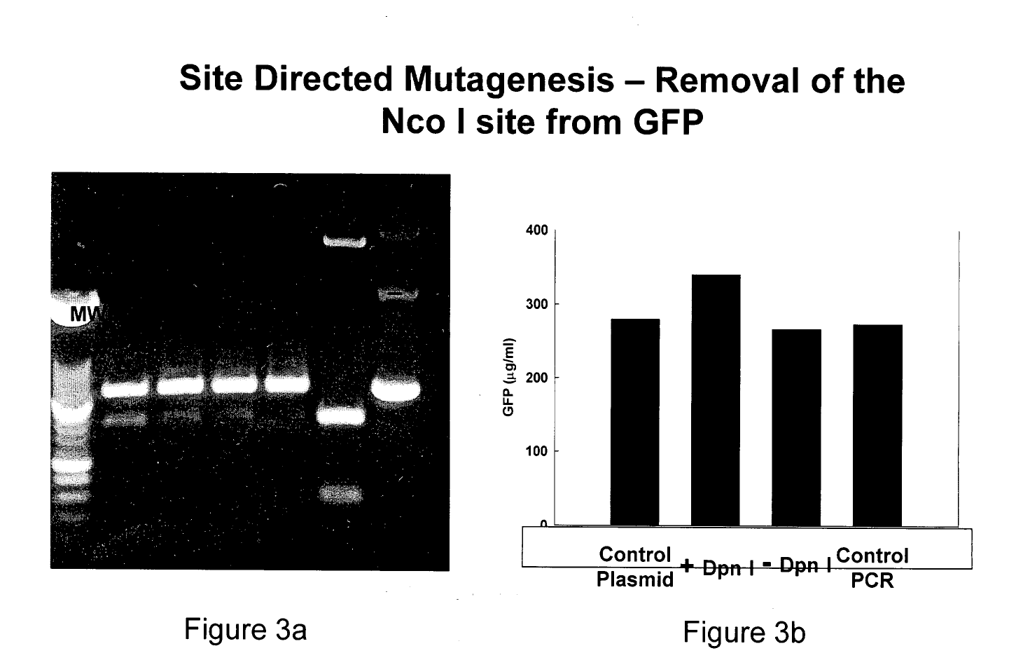

Generation of a mutagenized green fluorescent protein with PCR based mutagenesis. A plasmid comprising the green fluorescent protein is amplified with primers that provide for a mutation in the coding sequence, resulting in the loss of a restriction site, as shown in FIG. 3A and 3B.

The mutagenesis reaction was performed as follows:

Mutagenesis Reaction:

5 μl 10×reaction buffer+

2 μl 25 ng/μl pIVEX2.3 GFP+

2 μl 125 ng GM81 (CTACCTGTTCCTTGGCCAACACTTG) +
2 μl 125 ng GM82 (CGTGTTGGCCAAGGAACAGGTAG) +

1 μl dNTP solution+

37 μl HOH+

1 μl PfuTurbo Thermostable DNA Polymerase

50 μl total volume

PCR cycles performed:

15 cycles: 95° C. for 30 seconds 55°C. for 30 seconds 68° C. for 12 minutes

Following the thermocycling the reaction was diluted with:

20 μl Buffer A (RMB)

130 μl HOH

200 μl total

In order to reduce background from the initial template, the reaction mixture was combined with the restriction enzyme Dpnl, which is specific for methylated DNA. The initial template plasmid, which is grown in a bacterial host, comprises methyl-A residues, and is susceptible to digestion with the enzyme. The amplification product is not methylated, and so is not cleaved.

The amplification reaction was divided into two tubes each containing approximately 100 μl. Nothing was added to tube #1. 10 Units (5 μl) of Dpn I was added to tube #2. Both tubes were incubated at 37° C. for 1 hour.

Generation of an expressible PCR Product. Following the 1 hour incubation with Dpnl, either 5 μl or 2 μl of reaction mix were diluted into the following buffer (The remainder of the mutagenesis reaction was frozen at −20° C.):

10 μl 10×Buffer (Expand High Fidelity RMB)

2 μl dNTP Mix

2 μl GM144 (GCGCGCGAGATCTCGATCCCGCGAAATTAATACGAC)

2 μl GM147 (GCGCGCGTATCCGGATATAGTTCCTCCTTTCAG)

83 μl HOH

1 μl Expand High fidelity enzyme

As a control, pIVEX2.3GFP was diluted into the buffer and subjected to the same cycling conditions.

100 μl total each reaction (5 total reactions):

| | | | | |
|---|---|---|---|---|
| Dpn I | Dpn I | Dpn I | Dpn I | 200 ng |
| Untreated | Untreated | Treated | Treated | pIVEX2.3GFP |
| 5 μl of crude | 2 μl of crude | 5 μl of crude | 2 μl of crude | as a |
| mutagenesis | mutagenesis | mutagenesis | mutagenesis | non-mutated |
| reaction | reaction | reaction | reaction | control |

PCR cycles:

15 cycles: 95° C. 30 seconds 55° C. 30 seconds 72° C. 1 minutes

Confirmation That the DNA Sequence was Changed by the Mutagenesis Reaction

Following PCR, 5 μl of each of the 3 reactions (Dpn treated, Dpn untreated and control) was diluted into:

12 μl HOH

2 μl 10×buffer H (RMB)

1 μl Nco I

20 μl total volume

The restriction digests were incubated at 37° C. for 1 hour. The restricted PCR products were subjected to agarose gel electrophoresis to confirm the presence of the mutation (the lack of the endogenous Nco I site). Uncut PCR product derived from the parental plasmid was included as control.

The most prevalent band after gel electrophoresis from the mutagenesis reaction is uncut by Nco I. Because the control PCR product is efficiently cut by Nco I this suggests that the mutagenesis reaction and PCR resulted in a predominately mutant DNA fragment. See FIG. 1a.

Confirmation of PCR product-directed protein production. 2 μl of each unrestricted PCR product was added to a 50 μl RTS100 HY reaction. GFP activity was assayed using a scanning spectrofluorophotometer. The results indicate nearly identical activity among all samples. This would be expected since this mutation does not alter the amino acid sequence of the encoded protein. See FIG. 3b.

### Example 2

11 PCR products with engineered restriction sites for cloning were digested with Nco I and Xma I and ligated into similarly digested pIVEX vectors. A negative control was employed where insert was not added.

Briefly a reaction was set up of:

1-3 μl of digested insert DNA

1-3 μl of digested pIVEX DNA

3-5 μl of sterile water QES to make each reaction 8 μl

2 μl of 5×DNA dilution buffer

to the mixed reactions was added:

10 μl 2×buffer

followed by 1 μl T4 DNA ligase

The reactions were allowed to proceed for 5 minutes at room temperature.

Following the 5 minute incubation, 2 μl of each ligation reaction was added to a PCR reaction containing:

10 μl 10×Buffer (Expand High Fidelity RMB)

2 l dNTP mix

| | |
|---|---|
| 2 l GM143 | GGGGGCGAGATCTCGATCCCGCGAAATTAATACGAC |
| 2 GM146 | GGGGGGGTATCCGGATATAGTTCCTCCTTTCAG |

81 μl sterile PCR grade water

1 μl Expand High fidelity enzyme (RMB)

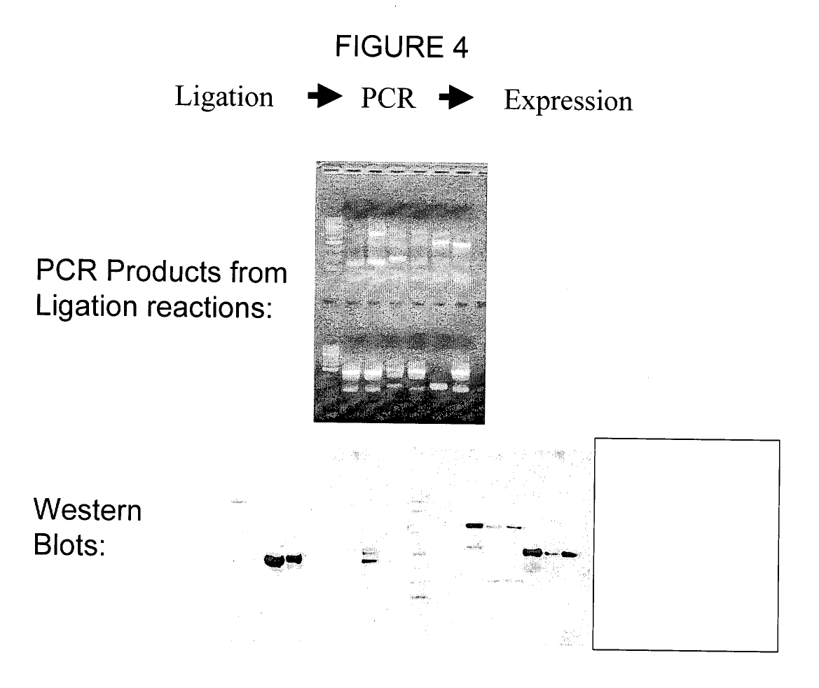

The PCR was performed for 30 cycles using 95 C. 1 minute/55 C. 1 minute/72 C. 1 minute. 5 μl of each PCR was subjected to agarose gel electrophoresis, and are shown in FIG. 4A. 10 μl of each PCR was further added to a cell-free transcription/translation reaction and incubated overnight at 30° C. The following day the results were analyzed by western blotting with anti-His antibodies (shown in FIG. 4B). These data show the in vitro expression of PCR products from a ligation reaction template.

### EXAMPLE 3

A recombinational cloning reaction was set up as follows using Gateway reagents and lambda recombinase (Invitrogen):

4 μl LR reaction buffer

2 μl pENTR-CAT

2 μl linearized pIVEX4.0-DEST

6 μl TE

After combining the above reagents, 4 μl of LR Clonase Enzyme Mix was added to initiate the reaction. The recombination reaction was allowed to proceed for 1.5 hours at 25 degrees C. 2 μl of Proteinase K was added. The reaction was terminated by incubation in the presence of proteinase K for 10 minutes at 37 degrees C.

Immediately following the termination of the recombination reaction, 1 μl of each recombination reaction was added to a PCR reaction containing:

10 μl 10×Buffer (Expand High Fidelity RMB)

2 μl dNTP mix

2 μl GM143 GGGGGCGAGATCTCGATCCCGCGAAATTAATACGAC
2 μl GM146 GGGGGGGTATCCGGATATAGTTCCTCCTTTCAG

82 μl sterile PCR grade water

1 μl Expand High fidelity enzyme (RMB)

5 μl of each PCR was subjected to agarose gel electrophoresis, as shown in FIG. 4A. 10 μl of each PCR was further added to a cell-free transcription/translation reaction and incubated overnight at 30° C. The following day the results were analyzed an HPLC-based activity assay for Choramphenicol acetyltransferase. The results of the PCR product derived from the recombinational cloning reaction was compared to a PCR product derived from the circular plasmid template or the circular plasmid template itself. No significant difference in activity was observed.

What is claimed is:

1. A method for streamlined high throughput screening and replication, the method comprising:
(a) amplifying an expressible portion of a replicatible initial template with a first and a second amplification primer;
(b) expressing said expressible portion in vitro to generate a polypeptide;
(c) screening said polypeptide for a property of interest;
(d) transforming a host cell with said replicatible initial template in the absence of an intervening cloning step.

2. The method according to claim 1, wherein said replicatible initial template is present in a recombination or ligation reaction.

3. The method according to claim 2, wherein said first primer and said second primer do not exponentially amplify linear reactants of said recombination or ligation reaction.

4. The method according to claim 1, wherein said expressible portion comprises a fragment of said replicatible initial template.

5. The method according to claim 1, wherein said expressible portion comprises the complete replicatible initial template.

6. The method according to claim 1, wherein said expressible portion comprises a promoter selected from the group consisting of a T7 promoter, T3 promoter, and SP6 promoter.

7. The method according to claim 1, wherein said expressible portion comprises a transcriptional termination sequence.

8. The method according to claim 1, wherein said expressible portion comprises an open reading frame comprising a genetic sequences of a pathogen; a genetic sequence encoding an enzyme; a genetic sequence encoding an antigen; or a genetic sequence involved in drug resistance.

9. The method according to claim 1, wherein said replicatible initial template comprises an origin of replication active in a plant cell, an animal cell, a fungal cell, or a bacterial cell.

10. The method according to claim 1, wherein said first primer comprises a GC clamp region at the terminus.

11. The method according to claim 9, wherein said GC clamp is from about 5 to about 10 nucleotides in length.

12. The method according to claim 1, said first primer hybridizes to a region of the initial template at, or upstream, of a promoter for said expressible portion, and wherein said first primer does not comprise to a complete promoter sequence.

13. The method according to claim 12, wherein said expressing step is performed on the reaction mixture from said amplifying step in the absence of a purification step.

14. The method according to claim 1, further comprising the step of reacting the product of said amplification step with an enzyme that specifically cleaves methylated DNA prior to said expressing step.

15. The method according to claim 1, wherein steps (a) to (d) are performed in parallel on a library of replicatible initial templates.

16. The method according to claim 15, wherein said library of replicatible initial templates are generated by mutagenesis of a sequence of interest.

17. A kit for streamlined high throughput screening and replication, comprising:
reagents for amplification of an expressible portion; reagents for in vitro transcription and translation; and host cells for transformation.
